(19) United States
(12) Patent Application Publication
(10) Pub. No.: US 2003/0055342 A1
(43) Pub. Date: Mar. 20, 2003

(54) Optical tomographic imaging apparatus

(75) Inventor: Masahiro Toida, Kaisei-machi (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/246,395

(22) Filed: Sep. 19, 2002

(30) Foreign Application Priority Data
Sep. 20, 2001 (JP) 286690/2001

(51) Int. Cl. A61B 6/00

(52) U.S. Cl. 600/478

(57) ABSTRACT

Low coherence light emitted from a light source is separated by a fiber coupler into a signal light, which is to be projected onto a target subject, and a reference light, which is to be modulated by a piezoelectric element. The signal light reflected from a predetermined depth of the target subject is synthesized with the reference light. The intensity of the interference light obtained thereby is detected by a balance differential detector, subjected to signal process by a signal processor, and displayed as a tomographic image on a display. The spectral width of the pulse light emitted from a pulse light source formed of an Er doped fiber is expanded in a zero dispersion fiber having a negative dispersion property, whereby the coherence length is shortened. The conventionally employed KTM Ti sapphire laser becomes unnecessary, and the light source becomes compact and inexpensive.

### BACKGROUND OF THE INVENTION

1. Field of the Invention

The present invention relates to an optical tomographic imaging apparatus for irradiating a measurement area with a signal light, which is a low coherence light, to obtain a tomographic image of the measurement area, and in particular to a an optical tomographic imaging apparatus that images the reflected light of the signal light projected onto the measurement area to obtain ultrafine structures data of the surface and depth portions of the measurement area.

2. Description of the Related Art

There are in use today optical tomographic imaging apparatuses that utilize a low coherence light to obtain a tomographic image of a target subject, which is a measurement area; wherein, more specifically, a tomographic image of, for example, sub-retinal ultrafine structures, is obtained by measuring, by use of a heterodyne detection, the intensity of a low coherence interference light.

According to an optical tomographic apparatus such as that described above, a low coherence light emitted from a Super Luminescent Diode (SLD) or the like is separated into a signal light and a reference light. The frequency of the reference light is shifted slightly by use of a piezoelectric element or the like. The signal light is projected onto the target subject, reflected from a predetermined depth of said target subject, and interference is caused between the reflected signal light and the reference light. The intensity of the interference light is measured by use of a heterodyne detection to obtain tomographic data of the target subject, wherein a movable mirror or the like disposed on the optical path of the reference light is caused to shift a microscopic amount to slightly change the length of said optical path, whereby the length of the optical path of the reference light and the length of the optical path of the signal light are caused to match, and the optical tomographic data of a predetermined depth of the target subject is obtained.

With regard to an optical tomographic apparatus such as that described above, because the tomographic data of a predetermined depth of a target subject is obtained, it is ideal that the interference is produced only when the length of the optical path of the reference light and the length of the optical path of the signal light are completely matched. However, in actuality, if the difference between the length of the optical path of the reference light and the length of the optical path of the signal light is less than the coherence length of the light source, interference is produced. That is to say, the resolution capability of the low coherence interference is dependent upon the coherence length of the light source.

Although the coherence length is influenced by the type of the light source, the oscillation mode, the noise level and the like, in general, a micro pulse light emitted from a pulse laser is used as the low coherence light. Regarding the micro pulse light, the pulse width and the coherence length thereof can be considered as being substantially proportional: for example, for a case in which a pulse laser having a center wavelength of 800 nm and a pulse width of 25 fs (10⁻¹⁵ sec) is employed, the coherence length is approximately 14 μm.

In recent years, the field of clinical medicine has seen a growing recognition of the utility of tomographic images of a measurement area of a patient. High resolution tomographic images not limited to those of the eyes, but of tissue having a higher light dispersion than the eyes have come to be desired. To this end, a high-output light source capable of emitting a low coherence light having a short coherence length is required. However, it is difficult to improve the output of an SLD. Also, because the spectral width is determined by the band gap, a problem arises in that it is difficult to broaden the spectral width, make the pulse width narrower, and shorten the coherence length. Also, it is in principle possible to obtain a high resolution tomographic image using the incoherent light emitted from a general purpose white light source or the like. However, in order to practically apply an optical tomographic imaging apparatus, it is desirable that the configuration thereof be of a fiber interferometer employing fiber, wherein a problem arises in that it is almost impossible to constrain an incoherent light at the diffractive limit thereof and guide the incoherent light within the fiber.

Therefore, apparatuses capable of obtaining high resolution tomographic images have been proposed, as described in, e.g., “Optics Letters” Vol. 21, No. 22 PP. 1839-41, by B. E. Bouma et. al 1996, wherein the light source is equipped with a Kerr-Lenz Modelocked (KLM) Ti: sapphire laser, which emits light having a pulse width of several fs, to realize a high-output, short pulse width low coherence light that is used as the signal light and the reference light, whereby high resolution tomographic images can be obtained.

Further, apparatuses capable of obtaining high resolution tomographic images have been proposed, as described in, e.g., “Optics Letters” Vol. 26, No. 9 PP. 608-610, by I. Hartl et. al 2001, wherein a Kerr-Lenz Modelocked (KLM) Ti: sapphire laser and photonic crystal fiber are provided as the light source, and high resolution tomographic images can be obtained utilizing the micro pulse light emitted from the above-described light source.

However, the light source of an optical tomographic imaging apparatus having a light source equipped with the KLM Ti: sapphire laser is cumbersome and of high cost. Further, the light source is difficult to use. From the standpoint of practicality, the cumbersomeness, high cost and complicated operation are problematic.

### SUMMARY OF THE INVENTION

The present invention has been developed in view of the forgoing problems, and it is an object of the present invention to provide an optical tomographic imaging apparatus for obtaining tomographic images utilizing low coherence light, wherein said apparatus is equipped with a light source that is not cumbersome, of high cost, or difficult to operate, and is capable of obtaining high resolution tomographic data.

The first optical tomographic imaging apparatus according to the present invention comprises:

a light source portion for emitting a low coherence light;

a wavelength separating and synthesizing means for separating the low coherence light into a signal light and a reference light, shifting the frequency of at least one of the signal light and the reference light so as to cause a difference between the frequency of the signal light and the frequency of the reference light, projecting the signal light onto the target subject, and causing interference between the signal light reflected from a predetermined depth of the target subject and the reference light; and

image detecting means for measuring the intensity of the interference light of the reflected light and the reference light to obtain, based on said measured intensity, a tomographic image of the target subject; wherein,

the light source comprises a lockedmode locked fiber laser for emitting pulse light, and a fiber for propagating said pulse light and which has a negative dispersion property within the wavelength band of said pulse light.

The second optical tomographic imaging apparatus according to the present invention comprises:

a light source portion for emitting a low coherence light;

a wavelength separating and synthesizing means for separating the low coherence light into a signal light and a reference light, shifting the frequency of at least one of the signal light and the reference light so as to cause a difference between the frequency of the signal light and the frequency of the reference light, projecting the signal light onto the target subject, and causing interference between the signal light reflected from a predetermined depth of the target subject and the reference light; and

image detecting means for measuring the intensity of the interference light of the reflected light and the reference light to obtain, based on said measured intensity, a tomographic image of the target subject; wherein,

the light source comprises a lockedmode locked semiconductor laser for emitting pulse light, and a fiber for propagating said pulse light and which has a negative dispersion property within the wavelength band of said pulse light.

Here, for a case in which the interference is caused between the reference light and the signal light after the frequency of at least one of the reference light or the signal light has been shifted, the phrase “shifting the frequency of at least one of the signal light and the reference light so as to cause a difference between the frequency of the signal light and the frequency of the reference light” refers to the shifting of the frequency of at least one of the reference light or the signal light so that a beat signal that repeats in a strong-weak cycle is produced at the difference frequency of the reference light and the signal light. Note that, “measuring the intensity of the interference light” refers to the measuring of the beat signal that repeats in a strong-weak cycle (interference light) produced at the difference frequency of the reference light and the signal light, by use of a heterodyne interferometer or the like, for example.

Further, “negative dispersion property” refers to the decreasing of the wavelength dispersion value (ps/nm/km) as the length of the wavelength increases. When the above-described pulse light is propagated through a fiber provided with a negative dispersion property such as that described above, the pulse width thereof is compressed. Further, the pulse light, of which the pulse width thereof has been compressed, is that which is emitted from the light source portion as a low coherence light.

A mode locked Er doped fiber laser can be used as the mode locked fiber laser. Further, a zero dispersion fiber or a photonic crystal fiber can be used as the fiber having a negative dispersion property.

Further, each of the optical tomographic imaging apparatuses described above can be further provided with an optical amplifying means for subjecting the reflected light to a light amplification process. A fiber amplifier such as an optical fiber amplifier, a fiber Raman amplifier or the like can be used as the optical amplifying means. Note that the referent of “optical amplifying means” is an optical amplifier utilizing an optical fiber to which rare earth elements or a colorant has been added, and which amplifies the light of the light signal; the optical fiber can be a glass type fiber formed of quartz, fluoride glass, tellurite glass or the like, or a plastic type optical fiber. Further, the referent of “fiber Raman amplifier” is a fiber amplifier that amplifies, by use of a induced Raman dispersion process, the light signal. Also, a semiconductor light amplifying means can be used as the optical amplifying means.

Still further, for cases in which the target subject is a portion of a living tissue, it is preferable that the wavelength of the low coherence light be in the range greater than or equal to 600 nm and less than or equal to 2000 nm.

In general, regarding the micro pulse light emitted from a pulse laser, because the reciprocal of the pulse width is substantially proportionate to the spectral width, the spectral width becomes broader corresponding to the narrowness of the pulse width, whereby the coherent length becomes short.

That is to say, if the pulse width of the low coherence light emitted from the light source of an optical tomographic imaging apparatus is made narrow, a high resolution tomographic image can be obtained.

According to the first optical tomographic imaging apparatus of the present invention: by propagating the pulse light, which is emitted from the lockedmode locked fiber laser, through a fiber having a negative dispersion property for the wavelength band of said pulse light, the pulse thereof is compressed and a pulse light having a narrow pulse width, that is, a low coherence light, can be obtained thereby. Accordingly, instead of the conventional light source, equipped with a micro pulse laser or the like, which is cumbersome, of high-cost and difficult to operate, low coherence light can be obtained by only providing a light source that is compact, inexpensive and easy to use, and the resolution capability of the low coherence interference can be improved.

According to the second optical tomographic imaging apparatus of the present invention: by propagating the pulse light, which is emitted from the lockedmode locked semiconductor laser, through a fiber having a negative dispersion property for the wavelength band of said pulse light, the pulse thereof is compressed and a pulse light having a narrow pulse width, that is, a low coherence light, can be obtained thereby. Accordingly, instead of the conventional light source, equipped with a micro pulse laser or the like, which is cumbersome, of high-cost and difficult to operate, low coherence light can be obtained by only providing a light source that is compact, inexpensive and easy to use, and the resolution capability of the low coherence interference can be improved.

Further, according to each of the optical tomographic apparatuses described above, if an Er doped fiber mode locked laser is used as the mode locked fiber laser, an even higher output pulse light can be easily obtained at the wavelength band employed in obtaining a tomographic image.

Still further, if a zero dispersion fiber is used as the fiber having a negative dispersion property, it becomes possible to obtain pulse width compression at a low cost. Also, if a photonic crystal fiber is used as the fiber having a negative dispersion property, a negative dispersion property can be obtained at a desired wavelength range.

In addition, for cases in which an optical amplifying means for amplifying the reflected light is to be provided, the optical amplifying means can be easily provided within the propagation path of the reflected light to amplify the signal intensity thereof. If a fiber amplifier such as an optical fiber amplifier, a fiber Raman amplifier or the like is used as the optical amplifying means, because it is possible to provide the amplifying fiber in a wound form, the amplifying fiber can be provided at a length capable of obtaining a desired amplification rate without requiring that the apparatus be made large and cumbersome, whereby the reflected light can be amplified at a high amplification rate by providing only a small sized fiber amplifier. Further, because low noise is a property of fiber amplifiers, the extremely faint reflected light can be accurately amplified. If a semiconductor amplifier is used as the optical amplifying means, the optical amplifying means can be made still further compact.

Further, if the target subject is a portion of a living tissue and the low coherence light has a wavelength within the range of greater than or equal to 600 nm and less than or equal to 2000 nm, because the signal light has favorable transmittance and dispersion properties with respect to the target subject, a desired tomographic image can be obtained.

### BRIEF DESCRIPTION OF THE DRAWINGS

FIG. 1 is a schematic drawing of the main portion of the first embodiment of the optical tomographic imaging means according to the present invention,

FIG. 2 is a graph illustrating the wavelength dispersion of a zero dispersion fiber,

FIG. 3 is a graph illustrating the wavelength dispersion of a pulse light,

FIG. 4 is a graph illustrating the spectral intensity of a low coherence light,

FIG. 5 is a schematic drawing of the main part of the light source portion according to the first embodiment,

FIG. 6 is a schematic drawing of the main part of a variation of the light source portion according the first embodiment,

FIG. 7 is a schematic drawing of the main portion of the second embodiment of the optical tomographic imaging means according to the present invention,

FIG. 8 is a graph illustrating the advantageous points of the optical amplifying means,

FIG. 9 is a schematic drawing of the main part of a variation of the optical amplifying means according the second embodiment, and

FIG. 10 is a schematic drawing of the main part of another variation of the optical amplifying means.

### DESCRIPTION OF THE PREFERRED EMBODIMENTS

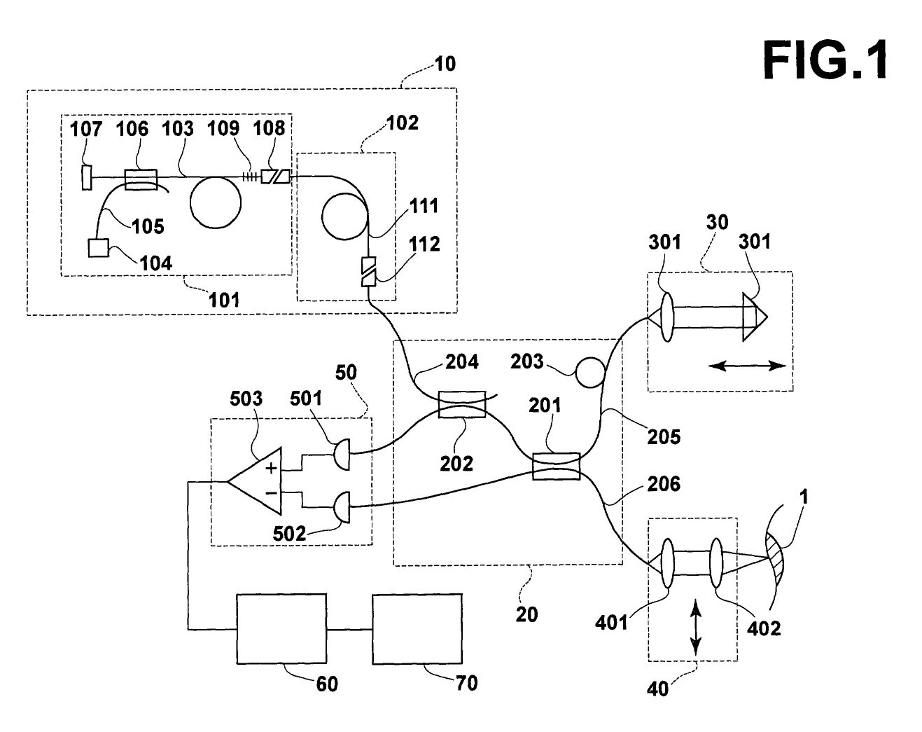

Hereinafter the first embodiment of the present invention will be explained with reference to FIG. 1. FIG. 1 is a schematic drawing of the main portion of the first embodiment of the optical tomographic imaging means according to the present invention.

The optical tomographic imaging apparatus according to the first embodiment comprises: a light source portion 10 that emits a low coherence light LI having a pulse width of approximately 10 fs, a center wavelength of 1.56 μm and a spectral width of approximately 800 nm; a fiber coupling optical means 20 for separating into a signal light L3 and a reference light L2 the low coherence light L1 emitted from the light source 10 and synthesizing the separated signal light L3 and reference light L2; an optical path extending portion 30, which is disposed on the optical path of the reference light L2, for extending the optical path of the reference light L2; an optical scanning portion 40 for scanning a target subject 1, which is a living tissue, with the signal light L3; a balance differential detecting portion 50 for detecting the intensity of the interference light L5 of the reference light L2 and the reflected light L4 reflected from a predetermined depth of the target subject; a signal processing portion 60 for obtaining, by performing a heterodyne detection process, the intensity of the reflected light L4 reflected from a predetermined depth of the target subject 1 from the intensity of the interference light L5 detected by the balance differential detecting portion 50, and converting said obtained intensity to an image signal; and an image display portion 70 for displaying as a tomographic image the image signal obtained by the image processing portion 60. Note that the fiber coupling optical system 20, the optical path extending portion 30 and the optical scanning portion 40 form the wavelength separating and synthesizing means, and the balance differential detecting portion 50 and the signal processing portion 60 form the image detecting means according to the present invention.

The light source portion 10 comprises a pulse light source portion 101 that emits a pulse laser light (hereinafter referred to as pulse light) having a wavelength of 1.56 μm, and a pulse compressing portion 102 for compressing the pulse width of the pulse light emitted from said pulse light source 101.

The pulse light source 101 is a mode locked Er doped fiber laser that emits a pulse light having a pulse width of approximately 100 fs, a center wavelength of approximately 1.56 μm and a spectral width of approximately 20 nm, comprising: an Er doped fiber 103; a excitation light semiconductor laser 104 for emitting the excitation light, which has a wavelength of 980 nm, to be guided into said Er doped fiber 103; a light guiding fiber 105 for guiding said excitation light; a fiber coupler 106 for introducing the excitation light into the Er doped fiber 103; a total reflecting mirror 107, which is a super-saturation absorbing mirror; and a light connector 108 for introducing the pulse light into the pulse compressing portion 102, which is a means further down the line in the processing sequence. Note that a fiber plug grating 109, which forms an output mirror, is formed at the output face of the Er doped fiber 103.

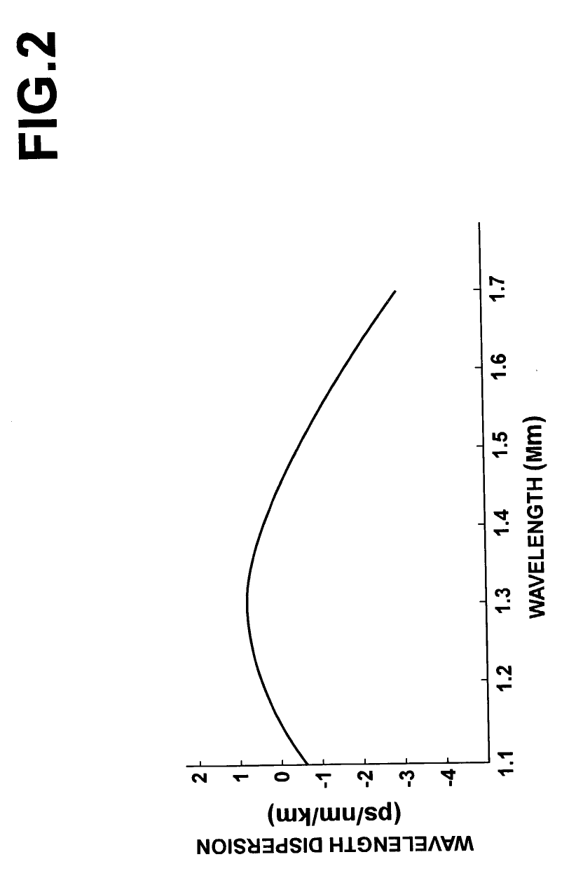

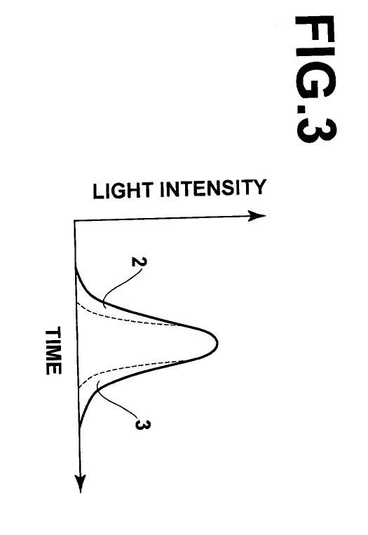

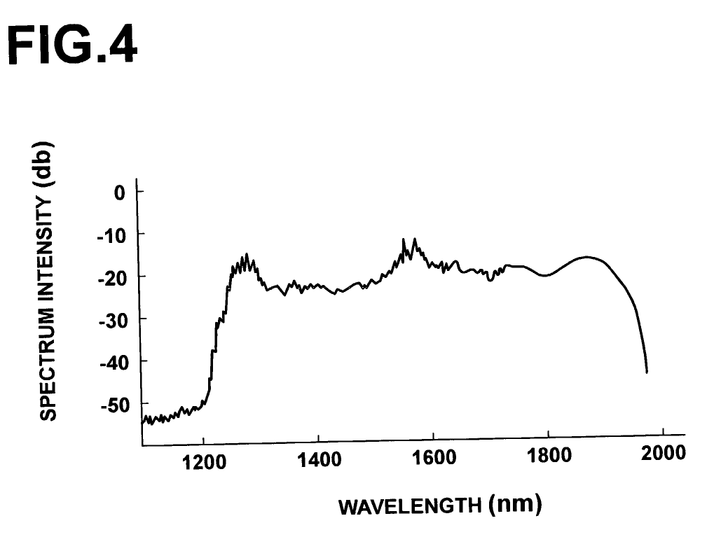

The pulse compressing portion 102, as shown in FIG. 2, comprises a zero dispersion fiber 111, which has a negative dispersion property in the vicinity of the wavelength of 1.56 μm, and a connector 112 for introducing the pulse light that has been compressed by the pulse compressing portion 102 into the fiber coupling optical system 20. In general, due to a self modulating phase effect, the interim waveform of the micro pulse light, which has a pulse width on the order of several fs and is emitted from the pulse laser, between the pulse times consists of a long wavelength component 3, followed by a short wavelength component 2, as shown in FIG. 3. When a micro pulse light of this type is propagated by a fiber having a negative dispersion property, the pulse width is compressed. Further, if the pulse width of the micro pulse light becomes narrow, because of the property of inexactness thereof, the spectral width becomes wider. Therefore, the compressed pulse light, which has been emitted from the pulse light source portion 101 and compressed by the pulse compressing portion 102 has a pulse width of approximately 10 fs and a spectral width of approximately 800 nm, as shown in FIG. 4.

The fiber coupling optical system 20 separates the low coherence light L1 emitted from the light source portion 10 into the reference light L2 and the signal light L3, and comprises: a fiber coupler 201 that synthesizes the reflected light L4 reflected from a predetermined depth of the target subject upon the irradiation thereof by the signal light L3 with the reference light L2 to obtain an interference light L5; a fiber coupler 202 disposed between the light source 10 and the fiber coupler 201; a piezoelectric element 203 for causing a slight shift in the frequency of the reference light; a fiber 204 that connects the light source 10 and the fiber coupler 202; a fiber 205 that connects, via the fiber couplers 201 and 202, the optical path extending portion 30 and the balance differential detecting portion 50; and a fiber 206 that connects, via the fiber coupler 201, the optical scanning portion 40 and the balance differential detecting portion 50. Note that the fibers 204, 205, and 206 are single mode optical fibers.

The optical path extending portion 30 comprises a lens 301 for converting the reference light L2 emitted from the fiber 205 to a parallel light and projecting the reflected reference light L2 into the fiber 205, and a prism 302 for changing the length of the optical path by the displacement thereof in the horizontal direction shown in FIG. 1.

The optical scanning portion 40 comprises a lens 401 and a lens 402 for displacing the signal light L3 in the vertical direction shown in FIG. 1 and projecting the reflected light L4 reflected by the target subject 1 into the fiber 206.

The balance differential detecting portion 50 comprises: a photo detector 501 and a photo detector 502 for detecting the intensity of the interference light L5; and a differential amplifier 503 for adjusting the input balance of the respective detection values of the photo detectors 501, 502, canceling out the noise component, the drift component and the like, and amplifying the difference between the value detected by the photo detector 501 and the value detected by the photo detector 502.

Next, the operation of the optical tomographic imaging apparatus according to the current embodiment will be explained. First, the pulse laser light, which has a pulse width of approximately 100 fs, a center frequency of 1.56 μm and a spectral width of approximately 20 nm, emitted from the mode locked Er ion doped fiber of the pulse laser light source 101 is projected, via the optical connector 108, into the pulse compressing portion 102.

In the pulse compressing portion 102, the pulse light is propagated by the zero dispersion fiber 111, the pulse thereof is compressed, and the pulse compressed pulse light is introduced into the fiber 204 of the fiber coupling optical system 20 via the optical connector 112. At this time, the pulse light has become the low coherence light L1 having a pulse width of approximately 10 fs, a center frequency of 1.56 μm and a spectral width of approximately 800 nm.

The low coherence light L1 transmitted by the fiber 204 is introduced into the fiber 205 at the fiber coupler 202, and is separated at the fiber coupler 201 into the reference light L2, which proceeds within the fiber 205 in the direction of the optical path extending portion 30, and the signal light L3, which proceeds within the fiber 206 in the direction of the optical scanning portion 40.

The reference light L2 is modulated by the piezoelectric element 203 disposed on the optical path thereof to produce a slight frequency difference Δf between the reference light L2 and the signal light L3.

The signal light L3 is projected through the lens 401 and the lens 402 of the optical scanning portion 40 and onto the target subject 1. From the signal light L3 entering the target subject 1, the reflected light L4 reflected from a predetermined depth of the target subject 1 is fed back via the lenses 401 and 402 to the fiber 206. The reflected light L4 fed back to the fiber 206 is synthesized in the fiber coupler 201 with the reference light L2 that has been fed back to the fiber 205, which is described below.

Meanwhile, after the reference light L2 has been modulated by the piezoelectric element 203, said reference light L2 is transmitted by the fiber 205, projected into the prism 302 via the lens 301 of the optical path extending portion 30, reflected by the prism 302, again transmitted by the lens 301, and is fed back to the fiber 205. The reference light L2 fed back to the fiber 205 is synthesized in the fiber coupler 201 with the above-described reflected light L4 that has been fed back to the fiber 206.

The reference light L2 and reflected light L4 that have been synthesized in the fiber coupler 201 are again synthesized along the same axis, and at a predetermined timing, interference is caused between said reflected light L4 and reference-light L2, whereby a beat signal is generated.

Because the reflected light L4 and the reference light L2 are low-coherence light of a short interference susceptibility distance, after the low coherence light has been separated into the signal light L3 and the reference light L2, if the length of the optical path of the signal light L3 (reflected light L4) up to the point at which said signal light L3 (reflected light L4) arrives at the fiber 201 is substantially the same as the length of the optical path of the reference light L2 up to the point at which said reference light L2 arrives at the fiber 201, both of said lights interfere with each other, said interference repeats in a strong-weak cycle according to the difference Δf between the frequencies of the reference light L2 and the signal light L3, and a beat signal is generated thereby.

The interference light L5 is separated in the fiber 201: one of the separated components thereof enters the photo detector 501 of the balance differential detector 50 after passing through the fiber 205; the other of the separated components thereof enters the photo detector 502 after passing through the fiber 206.

The photo detectors 501 and 502 detect the signal strength of the beat signal from the interference light L5, and the differential amplifier 503 obtains the difference between the detection value of the photo detector 501 and the detection value of the photo detector 502 and outputs said difference to the signal processing portion 60. Note that because the differential amplifier 503 is provided with a function for adjusting the balance of the direct current component of the value input thereto, even in a case, for example, in which drift occurs in the low coherence light emitted from the light source portion 10, by amplifying the difference after adjusting the balance of the direct current component, the drift component is cancelled out, and only the beat signal is detected.

Note that at this time, when the prism 302 is moved in the direction of the light axis (the horizontal direction appearing in FIG. 1), the length of the optical path of the reference light L2 up to the point at which said reference light L2 arrives at the fiber 201 changes. Therefore, because the length of the optical path of the signal light L3 (reflected light L4) that interferes with the reference light L2 also changes, the depth at which the tomographic data of the target subject 1 is obtained also changes.

According to the operation described above, after the tomographic data of a desired depth from a predetermined point on the surface of a target subject 1 has been obtained, the entry point of the signal light L3 is moved by the slight movement of lens 401 and the lens 402 of the optical scanning portion 40 in the vertical direction shown in FIG. 1, and the tomographic data is obtained at a predetermined depth in the same way. By repeating the above-described operation, the tomographic data of the target subject 1 can be obtained.

The signal processing portion 60 performs a heterodyne detection to detect the intensity of the reflected light L4 reflected from a predetermined depth of the target subject 1 from the intensity of the interference light L5 detected by the balance differential detecting portion 50, converts the obtained intensity of the reflected light L4 to an image signal, and displays a tomographic image on the image display portion 70.

In this fashion, because the low coherence light L1 is generated by compressing, by use of the pulse compressing means 102, the pulse of the pulse laser light emitted from the pulse light source portion 101 formed by the mode locked Er doped fiber laser, the light source portion 10 is made inexpensive, compact and easy to operate. Further, because a zero dispersion fiber is used in the pulse compressing portion 102, pulse compression can be performed at a low cost.

Further, the low coherence light L1 emitted from the light source portion 10 becomes a low coherence light having a pulse width of approximately 10 fs, a spectral width of approximately 800 nm, and a coherence length of approximately 3 μm. That is to say, the resolution of the low coherence interference becomes 3 μm.

Therefore, without using the conventionally employed cumbersome, high cost and difficult to operate light source that has been equipped with a micro pulse laser or the like, the resolution of the low coherence interference can be improved.

Further, because the pulse width of the pulse light emitted from the pulse light source 101 is approximately 100 fs, the pulse light can be efficiently compressed to a desired pulse width.

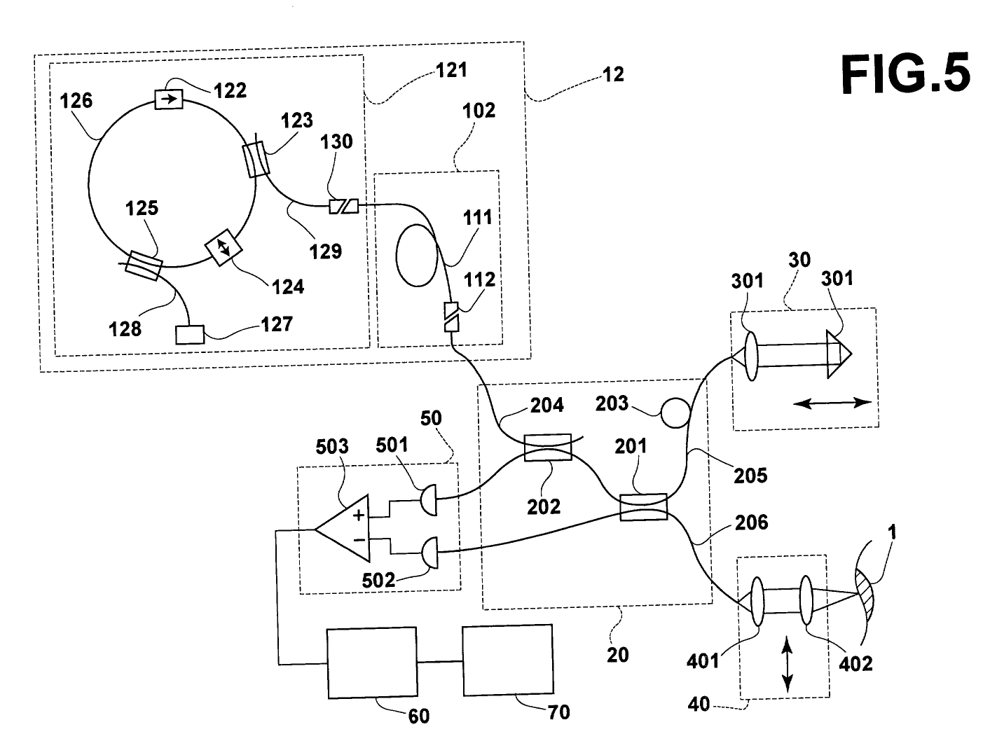

Note that as a variation on the current embodiment, an optical tomographic imaging apparatus employing a light source portion 12 comprising a pulse light source portion 121 utilizing a mode locked fiber ring laser, and a pulse compressing portion 102 as shown in FIG. 5 can also be considered. The pulse light source 121 comprises: a mode locked fiber ring laser formed of a polarization dependent isolator 122 an output coupler 123 a rotating polarization element 124, a synthesizing coupler 125, an Er doped fiber 126; an excitation light semiconductor laser 127 that emits an excitation light having a wavelength of 1.48 μm, and a fiber 128 for guiding the excitation light; an output fiber 129; and an optical connector 130. The micro pulse light, which has a pulse width of approximately 100 fs, a spectral width of approximately 20 nm, and a center wavelength of approximately 1.56 nm, emitted from the pulse light source 121 is outputted through the optical connector 130 to the pulse compressing portion 102, propagated by the zero dispersion fiber 111 wherein the pulse thereof is compressed, and emitted through the optical connector 112 into the fiber 204 of the fiber coupling optical system 20. At this time, the pulse light has become low coherence light having a pulse width of approximately 10 fs and a spectral width of approximately 800 nm. The specifics of the configuration and operational principles of the mode locked fiber ring laser are described in “Laser Research” Vol. 27, No. 11, pp. 756-61, by Masahiro Nakazawa et. al, 1999. Regarding fiber ring lasers, because oscillation is possible utilizing the maximum limit of the advantageous band width, high output pulse light having a short pulse can be easily obtained by use of a compact, inexpensive, and easy to operate light source.

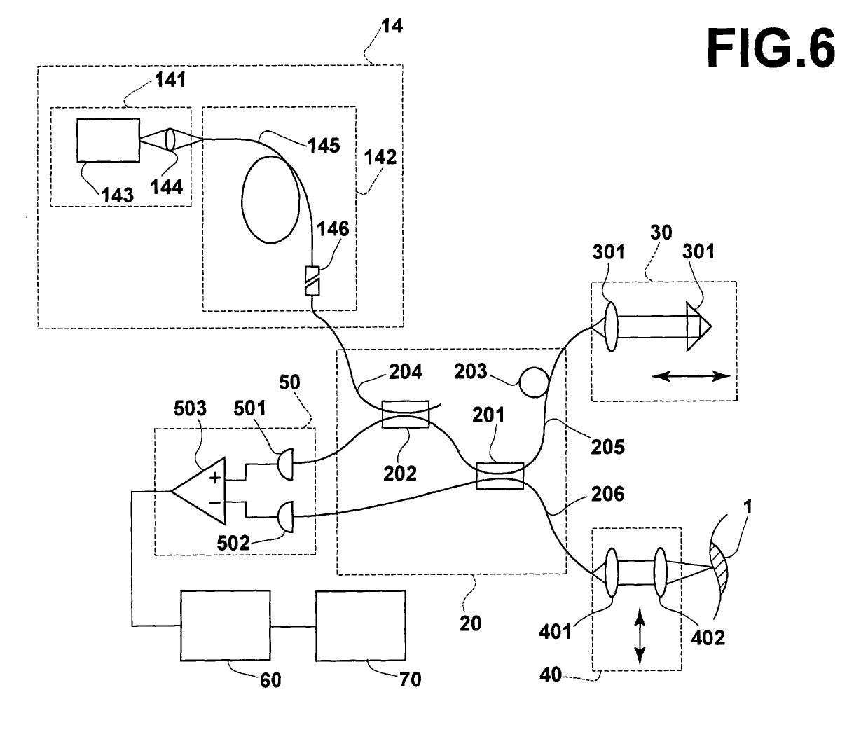

Further, as another variant on the current embodiment, an optical tomographic imaging apparatus employing a light source portion 14 instead of the light source 10, comprising a pulse light source portion 141, which employs a mode locked semiconductor laser, and a pulse compressing portion 142, as shown in FIG. 6. The pulse light source 141 comprises: a mode locked semiconductor laser 143 and a focusing lens 144 for introducing the pulse light emitted from the mode locked semiconductor laser into the pulse compressing portion 142. Further, the pulse compressing portion 142 comprises a photonic crystal fiber 145 having a zero dispersion property in the vicinity of the approximately 800 nm wavelength, and an optical connector 146. The specifics of the configuration and operational principles of the mode locked semiconductor laser are described in “Laser Research” Vol. 27, No. 11, pp. 750-55, by Hiroyuki Yokoyama, 1999. Further, the specifics of the configuration and operational principles of the photonic crystal fiber are described in “Optics Letters” Vol. 25, No. 1, pp. 25-7, by Andrew J Stenz et. al, 2001. Because the structural dispersion value of the photonic crystal fiber can be selected, a negative dispersion property can be realized in a desired wavelength range. Note that this type of photonic crystal fiber can also be combined with the pulse light source portion 101 or the pulse light source portion 121 to form the light source portion.

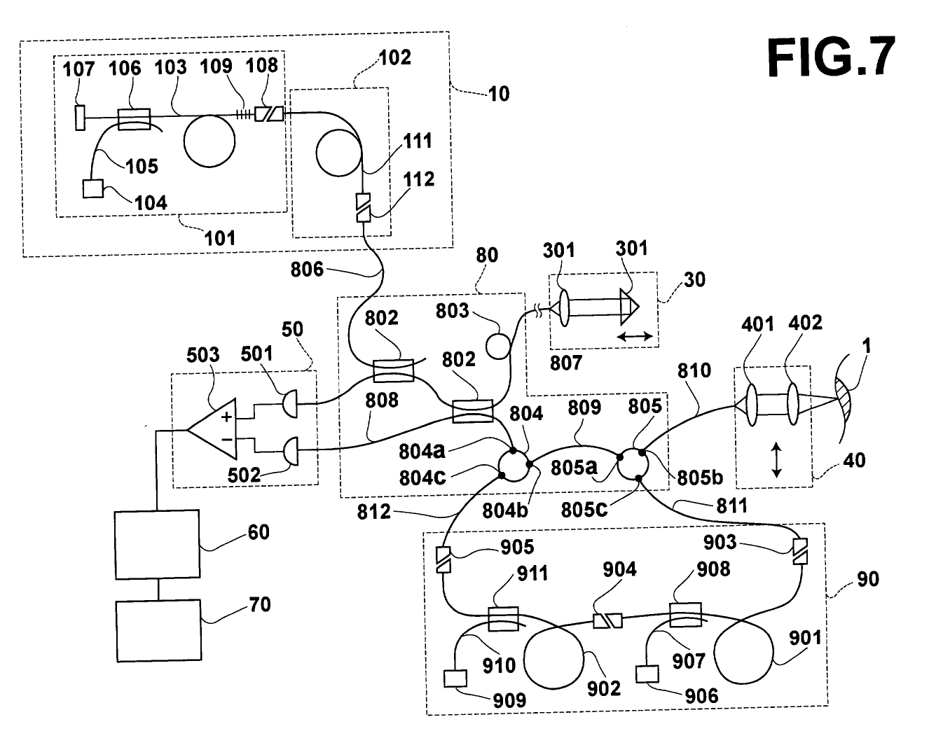

Next, the second embodiment of the present invention will be explained with reference to FIG. 7. FIG. 7 is a schematic drawing of the main portion of the second embodiment of the optical tomographic imaging means according to the present invention.

Note that elements in common with the first embodiment shown in FIG. 1 are likewise labeled, and in so far as it is not particularly required, further explanation thereof is omitted.

The optical tomographic imaging apparatus according to the second embodiment comprises: a light source portion 10 that emits a low coherence light L1 having a center wavelength of 1.56 μm and a spectral width of approximately 800 nm; a fiber coupling optical means 80 for separating into a signal light L3 and a reference light L2 the low coherence light L1 emitted from the light source portion 10 and synthesizing the reflected light L4 and reference light L2; an optical path extending portion 30, which is disposed on the optical path of the reference light L2, for extending the optical path of the reference light L2; an optical scanning portion 40 for scanning a target subject 1, which is a living tissue, with the signal light L3; an optical amplifier portion 90 formed by an optical fiber amplifier for amplifying the reflected light L4 reflected from a predetermined depth of the target subject 1 upon the irradiation thereof by the signal light L3; a balance differential detecting portion 50 for detecting the intensity of the interference light L5 of the reference light L2 and the amplified reflected light L4′; a signal processing portion 60 for obtaining, by performing a heterodyne detection process, the intensity of the reflected light L4 reflected from a predetermined depth of the target subject 1 from the intensity of the interference light L5 detected by the balance differential detecting portion 50, and converting said obtained intensity into an image signal; and an image display portion 70 for displaying as a tomographic image the image signal obtained by the image processing portion 60.

The fiber coupling optical system 80 comprises: a fiber coupler 801 that separates the low coherence light L1 emitted from the light source portion 10 into the reference light L2 and the signal light L3, and synthesizes the reflected light L4′, which is the signal light L3 that has been reflected from a predetermined depth portion of the target subject 1, and the reference light L2 to obtain an interference light L5; a fiber coupler 802 disposed between the light source 10 and the fiber coupler 801; an optical circulator 804, which has three ports, for transmitting the signal light L3 and the reflected light L4′ between the ports; an optical circulator 805, which has three ports, for transmitting the signal light L3 and the reflected light L4 between the ports; a piezoelectric element 803 for causing a slight shift in the frequency of the reference light L2; a fiber 806 that connects the light source 10 and the fiber coupler 801; a fiber 807 that connects, via the fiber couplers 801 and 802, the optical path extending portion 30 and one of the inputs of the balance differential detecting portion 50; a fiber 808 that connects, via the fiber couplers 801, the light circulator 804 and the other of the inputs of the balance differential detecting portion 50; a fiber 809 that connects the optical circulator 804 and the optical circulator 805; a fiber 810 that connects the optical circulator 805 and the optical scanning portion 40; a fiber 811 that connects the optical circulator 805 to the optical connector 903 of the optical amplifier portion 90, which is described below; and a fiber 812 that connects the optical circulator 804 and the optical coupler 902 via the fiber coupler 907. Note that the fibers 806 through 811 are single mode optical fibers.

The optical amplifier portion 90 comprises the following elements which function as described in an excitation state wherein signal light has been introduced thereto: an Er doped optical fiber 901 and a Pr doped optical fiber 902 for amplifying the signal light; an optical connector 903 for connecting the fiber 811 and the Er doped optical fiber 901; an optical connector 904 for connecting the Er doped optical fiber 901 and the Pr doped optical fiber 902; an optical connector 905 for connecting the Pr doped optical fiber 902 and the fiber 812; an excitation semiconductor laser 906 for emitting an excitation light L6, which has a wavelength of 980 nm, supplied to an Er doped fiber 901; an optical fiber 907 for guiding the excitation light L6; a fiber coupler 908 for introducing the excitation light L6 guided by said fiber 907 into the Er doped fiber 901; an excitation semiconductor laser 909 for emitting an excitation light L7, which has a wavelength of 1017 nm, supplied to a Pr doped fiber 902; an optical fiber 910 for guiding the excitation light L7; and a fiber coupler 911 for introducing the excitation light L7 guided by said fiber 910 into the Pr doped fiber 902.

The Er doped fiber 901 is formed of an Er doped fiber having an Er doped core, which can be advantageously used in the wavelengths near 1.56 μm; said optical fiber is provided in a wound state. The Pr doped fiber 902 is formed of an Pr doped fiber having an Pr doped core, which can be advantageously used in the wavelengths near 1.3 μm; said optical fiber is also provided in a wound state.

Next, the operation of the optical tomographic imaging apparatus according to the current embodiment will be explained. First, the low coherence light L1, which has a center frequency of 1.56 μm, emitted from the light source portion 10 is projected into the fiber 806.

The low coherence light L1 transmitted by the fiber 806 is introduced into the fiber 807 at the fiber coupler 802, and is separated at the fiber coupler 801 into the reference light L2, which proceeds within the fiber 807 in the direction of the optical path extending portion 30, and the signal light L3, which proceeds within the fiber 808 in the direction of the optical circulator 804.

The reference light L2 is modulated by the piezoelectric element 803 disposed on the optical path thereof to produce a slight frequency difference Δf between the reference light L2 and the signal light L3.

The signal light L3 enters the port 804a of the optical circulator 804, is projected from the port 804b into the fiber 809, enters the port 805a of the optical circulator 805, is projected from the port 805b into the fiber 810, and is projected through the lens 401 and the lens 402 of the optical scanning portion 40 onto the target subject 1.

Of the signal light L3 entering the target subject 1, the reflected light L4 thereof reflected from a predetermined depth of the target subject 1 is fed back, via the lenses 401, 402, to the fiber 810. The reflected light L4 fed back to the fiber 810 enters the port 805b of the optical circulator 805, and is projected into the fiber 811 from the port 805c. The reflected light L4 that has entered the optical amplifying portion 90 from the fiber 811 is amplified by the optical amplifying portion 90 to obtain the amplified reflected light L4′, and the reflected light L4′ is synthesized in the fiber coupler 801 with the reference light L2 that has been fed back to the fiber 807. Note that the details of the operation of the optical amplifying portion 90 will be described below.

Meanwhile, the reference light L2 modulated by the piezoelectric element 803 is transmitted by the fiber 807, projected into the prism 302 via the lens 301 of the optical path extending portion 30, reflected by the prism 302, again transmitted by the lens 301, and fed back to the fiber 807. The reference light L2 fed back to the fiber 807 is synthesized in the fiber coupler 801 with the above-described reflected light L4′.

The reference light L2 and reflected light L4′ that have been synthesized in the fiber coupler 801 are again synthesized along the same axis. Interference is caused between said reflected light L4′ and reference-light L2 at a predetermined timing to form the interference light L5, whereby a beat signal is generated.

The interference light L5 is separated in the fiber 801. One of the separated components thereof enters the photo detector 501 of the balance differential detector 50 after passing through the fiber 807. The other of the separated components thereof enters the photo detector 502 after passing through the fiber 808.

The photo detectors 501 and 502 detect the signal strength of the beat signal from the interference light L5, and the differential amplifier 503 obtains the difference between the detection value of the photo detector 501 and the detection value of the photo detector 502 and outputs said difference to the signal processing portion 60. The signal processing portion 60 performs a heterodyne detection to obtain the intensity of the reflected light L4 from the intensity of the interference light L5 detected by the balance differential detecting portion 50, converts the obtained intensity to an image signal, and displays the image signal obtained thereby as a tomographic image on the display portion 70.

Note that when the prism 302 is moved in the direction of the light axis (the horizontal direction appearing in FIG. 1), the length of the optical path of the reference light L2 up to the point at which said reference light L2 arrives at the fiber 801 changes. Therefore, because the length of the optical path of the signal light L4′ (reflected light L4) that interferes with the reference light L2 also changes, the depth at which the tomographic data of the target subject 1 is obtained also changes.

After the operation described above is repeatedly performed and the tomographic data of a desired depth from a predetermined point on the surface of a target subject 1 has been obtained, the entry point of the signal light L3 is moved slightly by the slight movement of lens 401 and the lens 402 of the light scanning portion 40 in the vertical direction shown in FIG. 4, and the tomographic data is obtained to a predetermined depth in the same way. By repeating the above-described operation, which is the same as that of the first embodiment, the optical tomographic data of the target subject 1 can be obtained.

Here, the details of the operation of the optical amplifying portion 90 will be explained. The excitation light L6, which has a wavelength of 980 nm, emitted from the excitation semiconductor laser 906 enters the Er doped fiber 901 through the fiber 908. As the excitation light L6 is propagated within the Er doped fiber 901, said excitation light L6 is absorbed by the Er with which the core of said fiber 901 has been doped. The Er absorbing the excitation light L6 the excitation light makes the transition from the base state to the excitation state. In this state, the reflected light L4 enters one end of the Er doped fiber 901. As said reflected light L4 is propagated within the core of said Er doped fiber, a light having the same phase as that of the reflected light L4 is inductively emitted, and the Er returns to the base state. By the repetition of this type of spontaneous emission, the amplified reflected light enters the Pr doped fiber 902 through the optical coupler 904.

In the Pr doped fiber also, the excitation light L7, which has a wavelength of 1017 nm, emitted from the excitation semiconductor laser 909 enters the Pr doped fiber 902 through the fiber 910. As the excitation light L7 is propagated within the Pr doped fiber 902, said excitation light L7 is absorbed by the Pr with which the core of said fiber 902 has been doped. The Pr absorbing the excitation light L7 the excitation light makes the transition from the base state to the excitation state. In this state, the reflected light enters one end of the Pr doped fiber 902. As said reflected light is propagated within the core of said Er doped fiber, a light having the same phase as that of the reflected light is emitted spontaneously, and amplified reflected light L4′ is emitted from the other end of the Pr doped fiber 902 and enters the fiber 812 through the optical coupler 905. Because the reflected light L4′ is the same phase signal as the amplified reflected light L4, tomographic image data can be obtained from the interference light generated by the interference of the reference light L2 and said reflected light L4′.

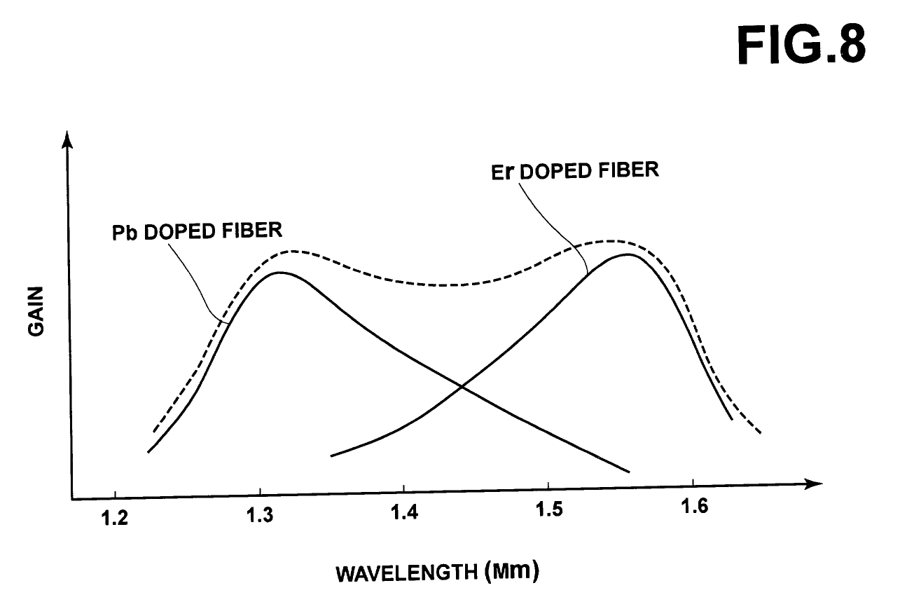

Note that the advantageous points of the optical amplifying portion 90 are shown by the broken line in FIG. 8. Because the advantageous points of the optical amplifying portion 90, the Er doped fiber 901 and the Pr doped fiber 902 become combined, a reflected light L4 having a wide spectral width such as that shown in FIG. 3 can be effectively amplified.

According to the operation described above, in addition to the effects obtained by the first embodiment, by measuring the intensity of the interference light L5 generated upon the interference of the reflected light L4′, which is the amplified reflected light L4 reflected from a predetermined depth of the target subject 1 upon the irradiation thereof by the signal light L3, and the reference light L2, effects whereby the intensity of the signal light can be maintained at an intensity that is safe for the measurement area of the patient, and optical tomographic image data having an improved S/N ratio can be obtained ca be realized. Further, with regard also to reflected light reflected from depths of the target subject 1 not capable of being obtained by conventional optical tomographic imaging apparatuses, by amplifying the reflected light L4 to obtain the reflected light L4′, because it becomes possible to detect the intensity of the interference light L5 generated by causing interference between the reflected light L4 and the reference light L2, the depth to which a tomographic image can be obtained is increased.

Further, because it is possible to provide the amplification optical fiber in a wound form, said amplification optical fiber can be made long and provided at a length obtaining a desired amplification rate without necessitating that the optical amplifying portion 90 be of a large, cumbersome size, the reflected light L4 can be amplified to a high amplification rate by the provision of only a small, compact optical amplifying portion 90. Further, because one property of the optical amplifying portion 90 is low noise, the extremely faint reflected light L4 can be accurately amplified.

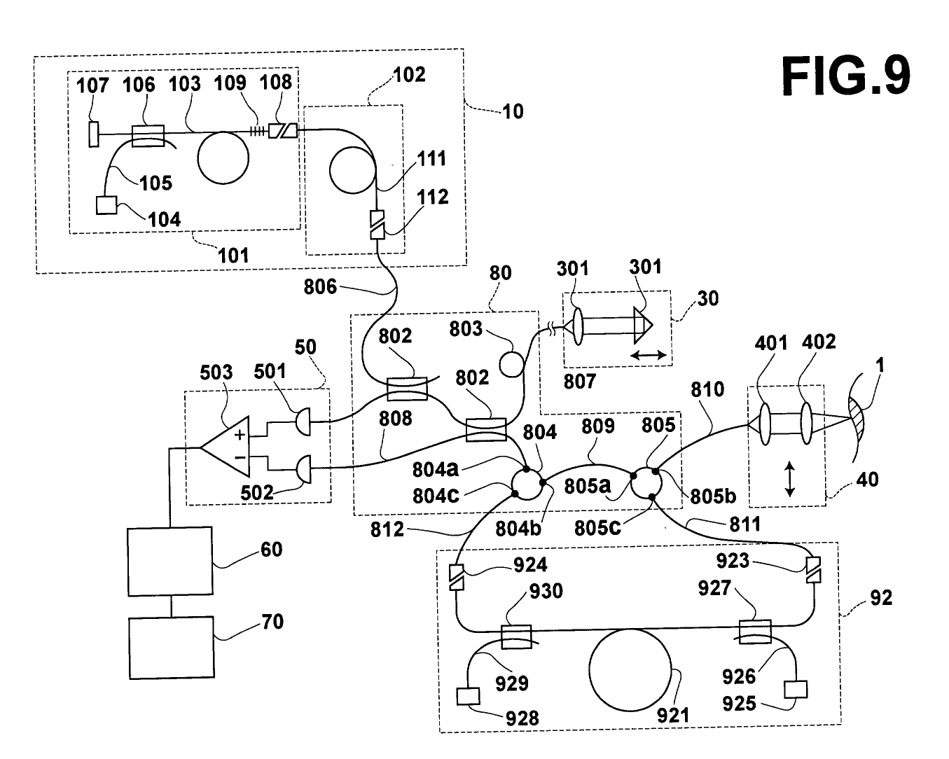

Still further, as a variant on the current embodiment, an optical amplifier 92 formed of a fiber Raman amplifier can be employed instead of the optical amplifying portion 90, as shown in FIG. 9. The optical amplifier 92 utilizes a stimulated Raman amplification process, and comprises: an optical fiber 921 for amplifying a light signal; an optical connector 923 and an optical connector 924 for connecting said optical fiber 921 to a fiber 811 and a fiber 812, respectively; a semiconductor laser 925 that emits an excitation light L6 having a wavelength of 1.48 μm; a fiber 926 for guiding the excitation light L6; a fiber coupler 927 for introducing the excitation light L6 guided by said fiber 926 into the optical fiber 921; a semiconductor laser 928 that emits an excitation light L7 having a wavelength of 1.017 μm; a fiber 929 for guiding the excitation light L7; and a fiber coupler 930 for introducing the excitation light L7 guided by said fiber 929 into the optical fiber 921. The specifics of the configuration and operational principles of a fiber Raman amplifier such as that described above are described in “0 Plus E” Vol. 21, No. 8, pp. 990-97, by Yoshihiro Emori et. al, 1999. According to the fiber Raman amplifier described above, by conveniently selecting the wavelength of the excitation light, light having a wavelength in a desired wavelength range can be amplified, whereby it is easy to realize amplification matched to the wavelength range of the low coherence light L1.

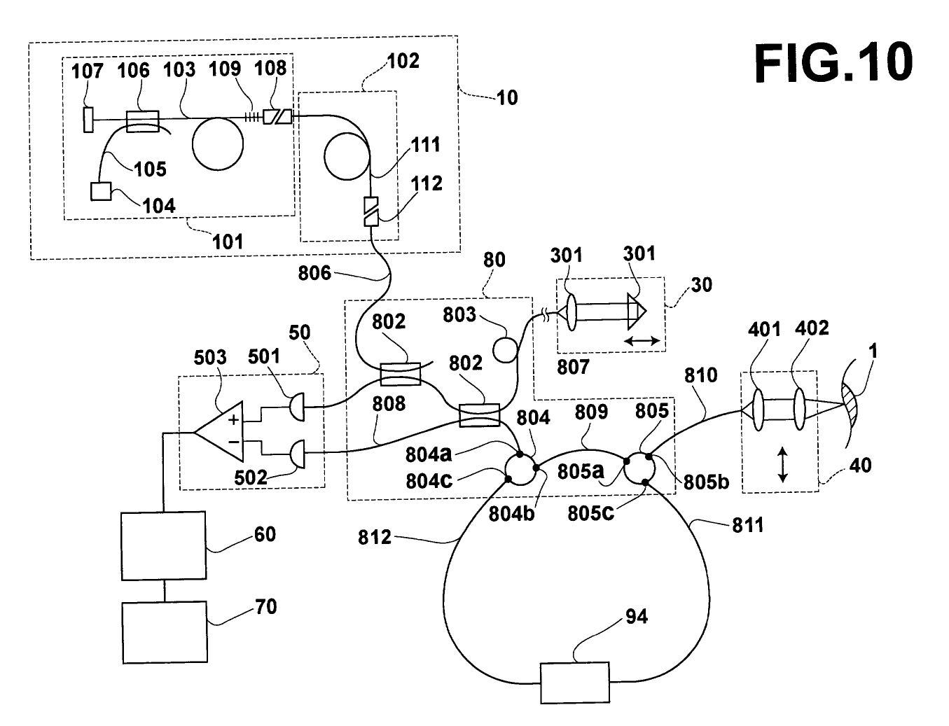

In addition, as another variant on the current embodiment, a semiconductor optical amplifier 94 can be used as the optical amplifier, as shown in FIG. 10. The specifics of the configuration and operational principles of a semiconductor optical amplifier are described in “0 Plus E” Vol. 21, No. 8, pp. 1006-1012, by Tatsuya Sasaki 1999. By employing the semiconductor optical amplifier 94, it becomes possible to make the optical amplifying portion extremely compact.

Note that although according to the second embodiment described above a mode locked Er doped fiber laser has been used as the pulse light source, and a zero dispersion fiber has been used as the pulse compressing portion, the present invention is not limited thereto. For example, a mode locked fiber ring laser, a mode locked semiconductor laser or the like can be used as the pulse light source. Further, it is preferable that a photonic crystal fiber or the like be employed as the pulse compressing portion.

Further, although according to each of the embodiments described above a piezoelectric element has been inserted in the optical path of the reference light and the frequency of the reference light has been shifted, the present invention is not limited thereto. It is also possible that the frequency of the signal light be shifted. Alternatively, the frequencies of both the reference light and the signal light can be shifted to provide a difference in both the respective frequencies thereof.

Still further, because the wavelength range of the low coherence light L1 is 1.2 μm to 2.0 μm, the signal light has favorable transmission and dispersion properties with respect to a living tissue target subject, whereby a desired optical tomographic image can be obtained.

What is claimed is:

1. An optical tomographic imaging apparatus comprising:
a light source portion for emitting a low coherence light,
a wavelength separating and synthesizing means for separating the low coherence light into a signal light and a reference light, shifting the frequency of at least one of the signal light and the reference light so as to cause a difference between the frequency of the signal light and the frequency of the reference light, projecting the signal light onto the target subject, and causing interference between the signal light reflected from a predetermined depth of the target subject and the reference light, and
image detecting means for measuring the intensity of the interference light of the reflected light and the reference light to obtain, based on said measured intensity, an optical tomographic image of the target subject, wherein,
the light source comprises a mode locked fiber laser for emitting pulse light, and a fiber for conveying said pulse light and which has a negative dispersion property within the wavelength range of said pulse light.

2. An optical tomographic imaging apparatus comprising:
a light source portion for emitting a low coherence light,
a wavelength separating and synthesizing means for separating the low coherence light into a signal light and a reference light, shifting the frequency of at least one of the signal light and the reference light so as to cause a difference between the frequency of the signal light and the frequency of the reference light, projecting the signal light onto the target subject, and causing interference between the signal light reflected from a predetermined depth of the target subject and the reference light, and
image detecting means for measuring the intensity of the interference light of the reflected light and the reference light to obtain, based on said measured intensity, an optical tomographic image of the target subject, wherein,
the light source comprises a mode locked semiconductor laser for emitting pulse light, and a fiber for conveying said pulse light and which has a negative dispersion property within the wavelength band of said pulse light.

3. An optical tomographic imaging apparatus as defined in claim 1, wherein
the mode locked fiber laser is a mode locked Er doped fiber laser.

4. An optical tomographic imaging apparatus as defined in any of the claims 1, 2, or 3, wherein
the fiber having a negative dispersion property is a zero dispersion fiber.

5. An optical tomographic imaging apparatus as defined in any of the claims 1, 2, or 3, wherein
the fiber having a negative dispersion property is a photonic crystal fiber

6. An optical tomographic imaging apparatus as defined in any of the claims 1, 2, or 3, further comprising an optical amplifying means for amplifying the reflected light.

7. An optical tomographic imaging apparatus as defined in claim 6, wherein
the optical amplifying means is a fiber amplifier.

8. An optical tomographic imaging apparatus as defined in claim 7, wherein
the fiber amplifying means is an optical fiber amplifier.

9. An optical tomographic imaging apparatus as defined in claim 7, wherein
the fiber amplifying means is a fiber Raman amplifier.

10. An optical tomographic imaging apparatus as defined in claim 6, wherein
the optical amplifying means is a semiconductor amplifier.

11. An optical tomographic imaging apparatus as defined in any of the claims 1, 2, or 3, wherein
the target subject is a living tissue portion, and
the wavelength of the low coherence light is in the range greater than or equal to 600 nm and less than or equal to 2000 nm.
